(19) United States
(12) Patent Application Publication
(10) Pub. No.: US 2006/0293561 A1
(43) Pub. Date: Dec. 28, 2006

(54) System and methods for intervertebral disc surgery

(76) Inventor: Eustaquio O. Abay II, Wichita, KS (US)

Correspondence Address: LATHROP & GAGE LC, 2345 GRAND AVENUE, SUITE 2800, KANSAS CITY, MO, 64108, US

(21) Appl. No.: 11/167,008

(22) Filed: Jun. 24, 2005

(51) Int. Cl. A61B 1/00

(52) U.S. Cl. 600/104

(57) ABSTRACT

This invention pertains to a surgical system for removing a damaged intervertebral disc and replacing it with a support system.

### FIELD OF THE INVENTION

The present invention relates to systems, instrumentation, and methods for removing damaged spinal discs, and for replacing said discs with an intervertebral support system.

### BACKGROUND OF THE INVENTION

The human spine is subject to a variety of ailments associated with the intervertebral disc. The intervertebral disc is the cornerstone of the joint complex that comprises the spinal motion segment. The disc functions to permit limited motion and flexibility, while maintaining segmental stability and absorbing and distributing external loads. The most common forms of intervertebral disc ailments are degeneration, bulging, herniation, thinning and degeneration with osteophyte formation.

The structure of the normal intervertebral disc includes a nucleus, composed primarily of proteoglycans and Type II collagen with a capacity to absorb and distribute load as well as an outer annulus with well-organized layer of Type I collagen that serve to stabilize the motion segment. The structure and function of the disc may be altered by processes including normal physiological aging, mechanical factors including trauma and repetitive stress, segmental instability of the spine, and inflammatory and biochemical factors.

Structural instability and dysfunction of the disc are important and significant causes of low back pain, and may be broadly encompassed by the term Degenerative Disc Disease. The pathology of the disc has an important role in the cause of low back pain. Treatment of low back pain may be most effective if disc function can be restored. Back pain is the most common ailment of the working-age adult, affecting over 4 million individuals each year in the United States, and weighing an economic burden on our health care system of up to $100 billion.

When a spinal disc ruptures, the outer part bulges and sometimes the inner nucleus escapes and can press on nerves or the spinal cord. The pressure on the nerves can cause significant pain and neurological symptoms such as numbness or tingling in the extremities; the arms or legs. If surgery is needed because of neurological symptoms, often the only way to take the pressure off the spinal cord or nerves is to entirely remove the disc. When this procedure is performed, something must fill the empty space, otherwise the bones keel forward and cause abnormal angling which, in themselves, may cause pain.

Most surgeons insert some form of bone into the space to fuse the vertebrae (bone) above and below the empty disc space. This often works very well in the short-term and can be done in many different ways that may include instrumentation such as cages, plates, and screws. Eventually the fusion becomes solid. However, there is a price to pay for such a technique. The levels above and below the fused or solid area are now forced to absorb more load as there is no spongy disc between the vertebrae. It is now known that up to 30% of discs above or below the level of the fusion wear out within 10 years and will require surgery. A stepladder effect can occur with multiple fusions over many years.

U.S. Pat. No. 6,436,143 teaches a method and apparatus for treating intervertebral disks by injecting thermoplastic materials into the damaged disks.

U.S. Pat. No. 6,558,390 teaches methods and apparatus for providing therapy to the spine including delivery of medicaments.

A way of removing discs and inserting a material into that space is needed that would retain the spine's mobility and share the physical loads exerted on the spine. It is not yet known if artificial discs can adequately achieve this aspect.

While most acute episodes of low back pain are self-limited and respond well to non-operative therapies, the management of chronic low back pain remains a difficult challenge for the non-operative and operative physician. Surgical management of chronic low back pain encompasses techniques including, intervertebral disc excision (discectomy), arthrodesis (fusion) of the spine using posterior, anterior, or combined approaches, intradiscal procedures including injections (epidurals), electrothermal exposure, and implantable neural stimulators and medication dispensers (spinal pumps).

The diversity of approaches and the variation observed in treatment strategies for low back pain indicates an absence of evidence-based support for any single method. While the rate of back surgeries including fusion of the spine increased by over 600% between 1979 and 1990, there remains no operative treatment that has yielded reliable and reproducible good results in patients affected by chronic low back pain. The common denominator is that there are no techniques directed toward the restoration of normal structure and function of the affected spinal motion segment, and of the disc at the center of that motion segment.

Motion within the musculoskeletal system is dependent upon functional joints. The goal of arthrodesis (fusion) is to eliminate motion of a segment or joint, and thereby relieve pain. The results of fusion include both failure to gain effective immobilization (pseudoarthrosis), and the induction of pathology in adjacent segments. For example, fusion of the hip for the treatment of degenerative joint disease is associated with a high rate of secondary low back and knee pathology. Total joint arthroplasty of the hip and knee has been recognized as one of the most effective surgical interventions of the 20th Century because of the efficacy of the procedure in relieving pain from an affected segment, while sparing adjacent segments or structures from pathologic stress or loads.

Fusion of the lumbar spine has increased at the highest rate of any spinal procedure in the last ten years. However, the indications, techniques, and results remain controversial and unclear. Intervertebral disc replacement is an attractive alternative to fusion of the spine with theoretical advantages that include restoration of segmental mobility, elimination of pain, and sparing of abnormal biomechanical forces on adjacent segments. Prosthetic devices of replacement of the intervertebral disc may be broadly divided into devices that replace the nucleus only, leaving the annulus and cartilaginous portions of the endplate intact, and devices that replace the entire intervertebral disc.

The artificial discs that are now being tested may ultimately provide the desired results. However, they have their inherent drawbacks, such as requiring radical surgical procedures, currently provide questionable results, and the medical procedures required to use these discs are expensive and time consuming.

Thus, an alternative approach for the surgical correction of these issues relating to spinal cord maladies would be of value. There is a need for a system for both open and percutaneous access to damaged vertebral discs in order that they may be replaced with a support system, wherein the vertebral disc surgery system minimizes overall patient trauma, and the resulting spacing between the vertebra can be precisely adjusted by the surgeon according to the needs of the patient.

### SUMMARY OF THE INVENTION

The present invention provides systems and methods for removing one or more damaged vertebral discs and replacing them with an artificial support system.

### BRIEF DESCRIPTION OF THE DRAWINGS

FIG. 1 illustrates a perspective view of the conduit and sharp tipped stylet.

FIG. 2 illustrates a perspective view of a posterior percutaneous entry of the conduit and stylet into the back of a patient.

FIG. 3 illustrates one view of the cutting tool in contact with an intervertebral disc.

FIG. 4 illustrates forceps used in removing the pulverized pieces of the intervertebral disc following utilization of the cutting tool.

FIG. 5 illustrates the support system still attached to the accompanying installation components.

FIG. 6 illustrates the intervertebral support system with the hollow tube being withdrawn following unthreading.

FIG. 7 illustrates the camera system according to the present invention.

FIG. 8 illustrates the scope holder employed in the camera system.

### DETAILED DESCRIPTION

The present invention is directed to systems and methods used to remove damaged, diseased and degenerated cervical, thoracic, and lumbar spinal discs and replacing them with a support system that can be positioned between the vertebra either through an open or percutaneous procedure.

The surgical system as utilized in the present invention employs percutaneous access to the damaged disc. The system and method of the invention may be exemplified as follows: (a) a conduit with at least one channel and a proximal and distal end; (b) a sharp tipped retractable stylet that when disposed within the at least one channel of the conduit is used to pierce the skin and underlying tissue of a patient and position the distal end of the conduit into the damaged disc; (c) a rotary cutting apparatus that can be slid into the at least one channel of the conduit for placement proximate the damaged disc and for pulverizing the disc into sections capable of being withdrawn through the conduit; (d) forceps that can be slid into the at least one channel of the conduit to facilitate grasping of and removal of the pulverized pieces of the vertebral disc; (e) a hollow rod with proximal and distal ends that can be slid into the at least one channel of the conduit for positioning and inflating a support system into the space previously occupied by the vertebral disc wherein the support system is detachable from the rod; and (f) a camera system with a probe slidably disposable through the at least one channel of the conduit for placement adjacent the disc, the probe connected to a viewing scope for use by the surgeon. The camera system further includes a scope holder for stabilizing the scope in position on the back of the patient.

The surgeon may insert the stylet into the conduit such that the sharp tip of the stylet is protruding. The surgeon then determines the direction and location of the percutaneous entry of the stylet positioning the stylet into the disc that is to be removed and replaced with an artificial disc.

The present invention provides for retraction of the stylet from the conduit and the insertion of a camera probe through one of the channels constructed into the conduit. In the adjacent channel is first inserted a cutting tool attached to the distal end of a drive shaft for pulverizing the nucleus pulposus and the annulus fibrosus of the vertebral disc to a size sufficiently small that it can be withdrawn through a channel of the conduit. The surgeon, utilizing the camera probe, directs the articulation and application of power to the cutting tool causing the damaged vertebral disc to be pulverized and capable of extraction in pieces through the conduit.

Once the disc pulverization process is complete, the surgeon extracts the cutting tool through the channel of the conduit and inserts in its place a forceps. The forceps is manually operated by the surgeon and is used to grasp pieces of the now pulverized vertebral disc. Once the pieces of the vertebral disc are grasped by the forceps they are retracted through the conduit. This process is repeated until all remnants of the disc are extracted from the space between the vertebrae.

Following removal of all vertebral disc remnants the surgeon then inserts a deflated support system into the area between the vertebrae with the assistance of a hollow insertion rod that is also inserted through a channel of the conduit. The hollow insertion rod is threaded at its distal end with male threads and the balloon has matching female threads into which the male threads are screwed. The matching reverse threads allow the deflated support system to be detachably secured to the rod. The rod is purposefully hollow to facilitate inflation of the support system with a physiologically inert polymeric material, such as methyl methacrylate. The support system can be inflated by the surgeon to the precise thickness and pressure according to the needs of the patient. The size of the balloon /deflated support system employed in this procedure may be modified to fit the nature of the intervertebral discs that are to be operated upon. For example, the balloon size employed for these surgical procedures performed on a cervical disc will be of a different size than that employed in a surgical procedure on a thoracic vertebral disc, which will be of a different size than that employed on a surgical procedure on a lumbar disc, and the like. Due to the nature of spinal injury and disease, the instant procedure will most likely be performed on diseased and damaged lumbar discs. This procedure corrects angulation deformities, such as kyphosis, scoliosis, or accentuated lordosis, while maintaining mobility. Previously, many patients were treated with spinal disc fusion and instrumentation, which typically would require additional surgeries in the future. These additional surgeries always increase the total cost of the procedure, as well. The nature of the instrumentation employed in these procedures will be the same.

As with the cutting tool procedure and the forceps procedure the deflated high strength support system and rod are inserted into a channel of the conduit and the support system's precise placement between the vertebrae is determined by the surgeon with the assistance of the camera system. Once the support system is properly positioned and pressurized and the inert material injected into the support system by the surgeon has adequately hardened, the hollow rod connected to the support system can be unscrewed and withdrawn through the conduit. The support system has a self sealing valve that prevents the release of any of the material utilized to pressurize the support system when the hollow rod is released from the support system.

In the event that more than one support system placed between the vertebrae is required to correct the patient's condition, the surgeon can introduce a second deflated support system into position following the procedures outlined above.

The support system employed in the present invention is typically inflatable, and is readily obtainable by one skilled in the art. The degree of inflation is determined by the nature of the operation and is readily manipulated by the practitioner.

The disc to be removed is typically diseased or otherwise damaged. An example of a diseased disc is one which has degenerated due to disc degeneration, which may be age-related. Damaged discs may include herniated discs. The disc to be removed may be cervical, thoracic, or lumbar in nature.

The disc that is diseased or damaged may be removed by any suitable means. Typically, the disc is either physically or chemically removed. If the disc is physically removed, the disc is typically surgically cut away from the existing placement, and is then removed. Sonication of the damaged or diseased disc may also be used, followed by the removal of the disc particles. Sufficient sonic energy is directed to the diseased or damaged disc to reduce the disc to smaller pieces, which may then be readily removed. The placement of the sonic energy source in the proximity of the damaged or diseased disc would typically rely on the conduit means discussed herein of the system of the present invention. Care would be employed so as to limit the effects of the sonic energy to that of the damaged or diseased disc. The removal of the disc pieces would then be also facilitated using the conduit means. Other suitable physical means for the removal of the diseased or damaged disc may be employed.

An example of chemical removal of the diseased or damaged disc includes enzymatic removal. The enzyme employed must be biocompatible with the existing structure. An example of enzymatic removal includes a process called chemonucleolysis, which is a medical procedure that involves dissolving the gelatinous cushioning material in an intervertebral disc by the injection of an enzyme, such as chymopapain. Chymopapain is derived from papaya. Care should be exercised to check for allergies to papaya in candidate patients. Surgical grade chymopapain may be obtained from any appropriate medical supply purveyor. Other suitable chemical means for the removal of the diseased or damaged disc may be employed.

The inert materials for injection into the support system may be formed of or include any suitable biocompatible materials. Examples of suitable biocompatible materials typically are those which are easy to work with, and which provide adequate support once placed into the support structure. The materials may be crosslinked prior to, during, or after placement within the support system in order to provide a more rigid structure once inserted into the support structure.

Suitable inert materials may include polymers and polymer blends such as acrylates, polyhydroxyalkanoates, and hydrogels. These substances typically possess controlled degradation rates, which may be determined according to the use of the inert material. The degradation rate is preferably determined under physiological conditions. An example of an acrylate is methyl methacrylate, and the like. Preferred for the practice of the present invention is methyl methacrylate.

Polyhydroxyalkanoates may also be employed, and include polymers selected from the group consisting of poly-4-hydroxybutyrate, poly-4-hydroxybutyrate-co-3-hydroxybutyrate, poly-4-hydroxybutyrate-co-2-hydroxybutyrate, and copolymers and blends thereof.

Hydrogels, which may be employed in the present invention, once solidified, may also exhibit mechanical as well as biocompatible properties that render them suitable for injection into the support structure. The hydrogel may be implanted into a support structure by injecting a thermogelling hydrogel comprising poly(N-isopropyl acrylamide) (PNIPAAm) copolymerized and/or blended with a second polymer. The thermogelling hydrogel is injected into the support structure as a liquid at room temperature, which then solidifies to form a solid implant as the hydrogel warms to physiological body temperature at the support structure site. Once solidified, the thermogelling hydrogels which may be employed in the present invention, exhibit adequate mechanical properties for use in support, as well as possessing biocompatibility properties in order to serve as a useful support material within the support structure.

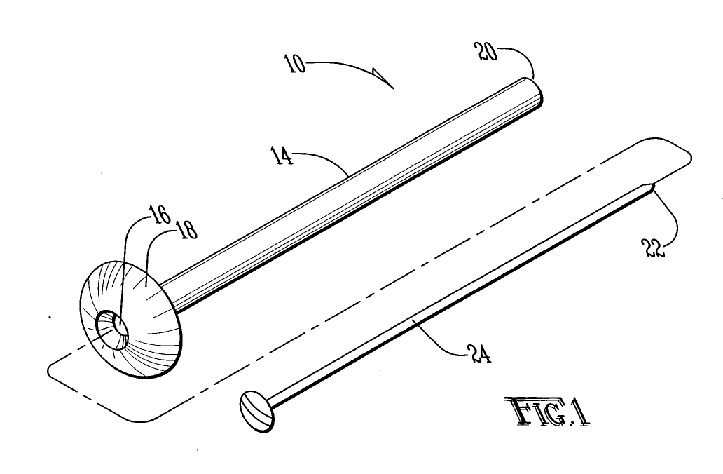

Specific aspects of the present invention include a system for removing a damaged vertebral disc and replacing it with an artificial support system. One aspect of the surgical system 10 is employed using percutaneous access to the damaged disc, as seen in FIG. 1, and includes a conduit 14 with at least one channel 16 through the conduit and a proximal end 18 and distal end 20.

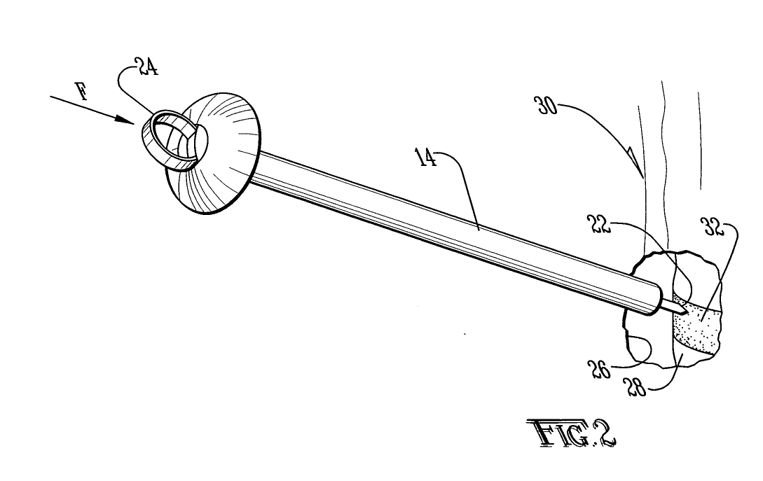

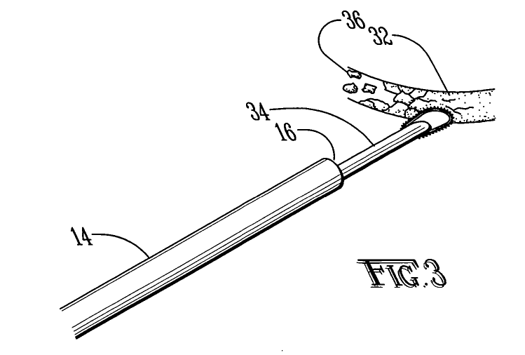

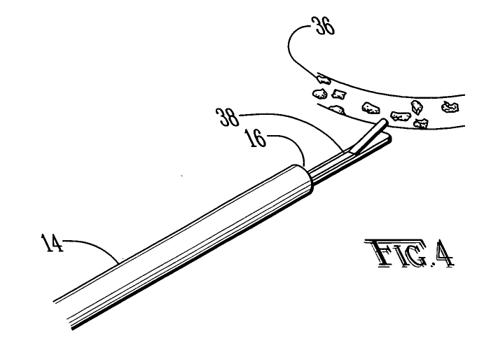

As seen in FIG. 2, a force F is applied by the surgeon to a sharp tipped 22 retractable stylet 24 that when disposed within the at least one channel 16 of the conduit 14 is used to pierce the skin 26 and underlying tissue 28 of a patient 30 and position the distal end 20 of the conduit 14 into the damaged disc 32. FIG. 3 shows a rotary cutting apparatus 34 that can be slid into the at least one channel 16 of the conduit 14 for placement proximate the damaged disc 32 that is used to pulverize the disc 32 into pieces 36 capable of being withdrawn through the conduit 14. Following the pulverization process and as seen in FIG. 4, a forceps 38 slidable through the at least one channel 16 of the conduit 14 is utilized to facilitate the grasping of and removal of the pulverized pieces 36 of the intervertebral disc 32 to clear the gap between the vertebra for insertion of the support system.

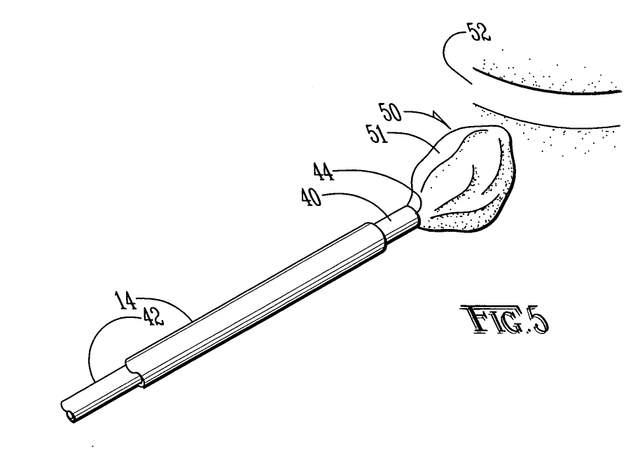

In conjunction with the foregoing and as seen in FIG. 5, a hollow rod 40 with proximal 42 and distal 44 ends is slid into the at least one channel 16 of the conduit 14 for positioning and inflating a support system 50 into the space 52 previously occupied by the vertebral disc 32 wherein the support system 50 is detachable from the hollow rod 40. As also seen in FIG. 5 the support system 50 is initially configured in a deflated or uninflated state that facilitates insertion of the support system through the channel 16 of the conduit. The support system 50 in its uninflated state is similar in configuration to a small balloon or pillow. The support system exterior 51 is typically constructed of a relatively inert material. Preferably the support system is constructed of a Teflon®-like material, or some other suitable material that is physiologically inert.

Following insertion of the support system 50 into the intervertebral space 52 and following proper orientation of the support system 50 between the vertebrae the attending surgeon proceeds to inflate, or pressurize, the support system with a biocompatible or physiologically inert material or combination of such materials having the mechanical strength capable of maintaining the desired intervertebral space 52. A preferred material for inflating the support system is methyl methacrylate; however, this is only exemplary of a material that may be utilized.

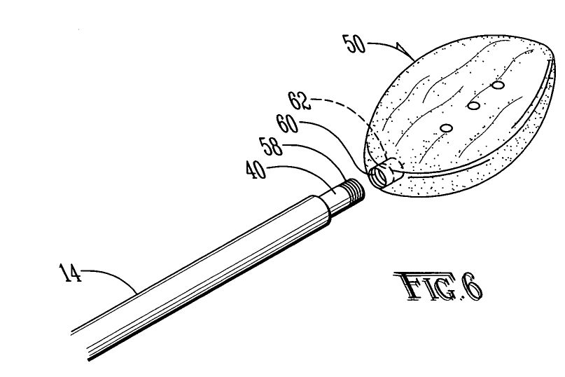

As seen in FIG. 6, once the support system 50 has been inflated and the material stabilizes in hardness, the surgeon unscrews the male threads 58 of the hollow rod 40 from the female threads 60 of the support system 50 and withdraws the hollow tube through the channel 16 of the conduit 14. The support system 50 is self sealing and the material forced into the support system cannot escape or leak because of a one-way valve 62 located proximate the female threads 60 within the support system 50.

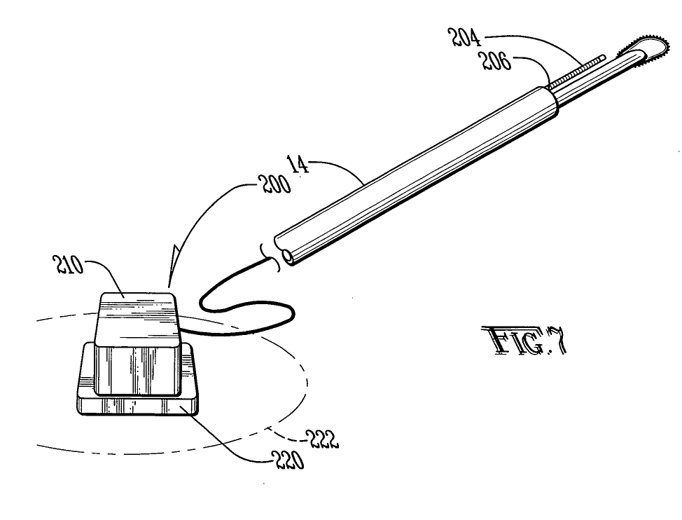

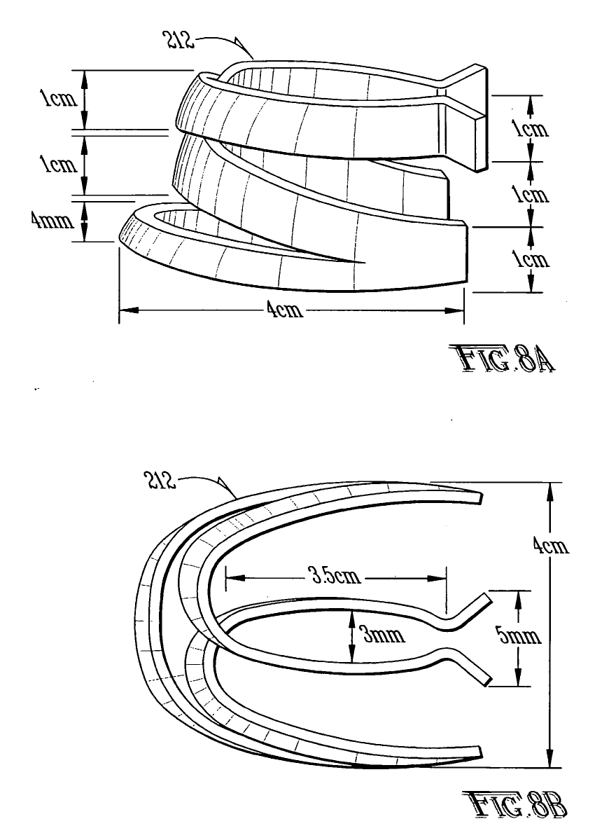

In a percutaneous procedure the surgeon must be able to view the process of pulverizing the damaged disc with the cutting tool, extracting the disc with the forceps and ultimately properly position the support system 50 between the adjacent vertebrae. As shown in FIG. 7, the surgeon typically employs a camera system 200 to facilitate the performance of the extremely intricate and detailed surgical procedures. The camera system 200 is positioned in proximity to the procedures being performed, and is typically employed with a probe 204 slidably disposable through a second channel 206 of the conduit 14 for placement adjacent the disc. The probe 204 is connected to a viewing scope 210 for use by the surgeon. The camera system 200 further includes a scope holder 212 for stabilizing the scope 210 in position on the back 220 of the patient 222. A view of the scope holder 212 is shown in FIG. 8.

If an open or anterior approach is selected to access the damaged intervertebral disc then the surgeon will proceed through the front of the patient to bypass or circumvent the internal organs to gain access to the spine. If this approach is selected then the instrumentation demands of the above referenced system are reduced. The surgeon need not employ the channel 14 and stylet 24 because percutaneous entry and posterior access to the intervertebral disc is not required. Likewise, the need for a camera system may be greatly reduced because viewing the process of pulverizing the disc and removing the disc remnants is simplified with an anterior approach.

What is claimed is:

1. A system for removal of a damaged or diseased intervertebral disc and replacement of the disc with a support structure, the system comprising:
(a) a conduit with at least one channel disposed therein;
(b) a removable stylet for placement within the at least one channel of the conduit to facilitate penetration of the skin and tissue covering the intervertebral disc to be removed;
(c) a cutting tool having a shaft with a distal end and a proximal end, and a cutting head secured to the distal end of the cutting tool, wherein the cutting tool is slidably disposable into the conduit and utilized to pulverize the intervertebral disc into segments sufficiently small to be withdrawn through a channel of the conduit;
(d) a forceps for grasping and retracting pulverized disc segments through the at least one channel of the conduit; and
(e) a hollow rod with a distal and proximal end, the distal end of the rod operatively configured to deliver the support structure for insertion through the at least one channel of the conduit in position between the vertebra,
wherein once in position a physiologically inert material is injected into the support structure through the hollow rod thereby causing the support structure to separate the adjacent vertebrae followed by subsequent detachment of the hollow rod from the artificial support structure and withdrawal of the rod through the at least one channel of the conduit.

2. The system of claim 1, further comprising a camera system, wherein said camera system comprises:
(a) a probe, and
(b) a scope,
wherein the probe is deliverable to the location of the damaged disc through the at least one channel of the conduit, the camera probe being capable of articulated by a user to facilitate viewing through the scope pulverization and removal of the disc segments and the positioning of the support structure.

3. The system of claim 2, wherein the camera system further comprises a scope holder, the scope holder comprising a base and an engagement portion wherein the engagement portion remains in biased contact with the scope and the base is disposed atop the back of the patient undergoing the spinal surgery.

4. The system of claim 1 wherein said inert material is selected from the group consisting of polymers and polymer blends.

5. The system of claim 4 wherein said polymer is selected from the group consisting of acrylates, polyhydroxyalkanoates, and hydrogels.

6. The system of claim 5 wherein said polymer is methyl methacrylate.

7. The system of claim 1 wherein said intervertebral disc is a cervical disc.

8. The system of claim 1 wherein said intervertebral disc is a thoracic disc.

9. The system of claim 1 wherein said intervertebral disc is a lumbar disc.

10. A system for removal of a damaged or diseased intervertebral disc and replacement of the disc with a support structure, the system comprising:
(a) a conduit with at least one channel disposed therein;
(b) a removable stylet for placement within the at least one channel of the conduit to facilitate penetration of the skin and tissue covering the intervertebral disc to be removed;
(c) a physical or chemical means for rendering said damaged or diseased disc into disc segments;
(d) a forceps for removing said disc segments through the at least one channel of the conduit; and
(e) a hollow rod with a distal and proximal end, the distal end of the rod operatively configured to deliver the support structure for insertion through the at least one channel of the conduit in position between the vertebra,
wherein once in position a physiologically inert material is injected into the support structure through the hollow rod thereby causing the support structure to separate the adjacent vertebrae followed by subsequent detachment of the hollow rod from the artificial support structure and withdrawal of the rod through the at least one channel of the conduit.

11. The system of claim 10, further comprising a camera system, wherein said camera system comprises:
(a) a probe, and
(b) a scope,
wherein the probe is deliverable to the location of the damaged disc through the at least one channel of the conduit, the camera probe being capable of articulated by a user to facilitate viewing through the scope pulverization and removal of the disc segments and the positioning of the support structure.

12. The system of claim 11, wherein the camera system further comprises a scope holder, the scope holder comprising a base and an engagement portion wherein the engagement portion remains in biased contact with the scope and the base is disposed atop the back of the patient undergoing the spinal surgery.

13. The system of claim 10 wherein said inert material is selected from the group consisting of polymers and polymer blends.

14. The system of claim 13 wherein said polymer is selected from the group consisting of acrylates, polyhydroxyalkanoates, and hydrogels.

15. The system of claim 14 wherein said polymer is methyl methacrylate.

16. The system of claim 10 wherein said physical means is selected from the group consisting of sonication and surgically cutting.

17. The system of claim 16 wherein said physical means is sonication.

18. The system of claim 16 wherein said physical means is surgical cutting.

19. The system of claim 10 wherein said chemical means is enzymatic treatment.

20. The system of claim 19 wherein said enzymatic treatment is chymopapain treatment.

21. The system of claim 10 wherein said intervertebral disc is a cervical disc.

22. The system of claim 10 wherein said intervertebral disc is a thoracic disc.

23. The system of claim 10 wherein said intervertebral disc is a lumbar disc.
